(19) United States
(12) Patent Application Publication
(10) Pub. No.: US 2026/0347033 A1
(43) Pub. Date: Nov. 5, 2026

(54) FEEDING STATION FOR THE DECONTAMINATION OF CONTAINERS

(71) Applicant: I.M.A. INDUSTRIA MACCHINE AUTOMATICHE S.P.A. IN SIGLA IMA S.P.A., OZZANO DELL'EMILIA (IT)

(72) Inventors: Filippo FURLOTTI, TRAVERSETOLO, Localita Cazzola (IT); Giacomo GUIDI, LONGIANO (IT); Michele FERRI, NOCETO (IT); Annalisa MALFATTO, PARMA (IT)

(21) Appl. No.: 19/103,268

(22) PCT Filed: Jul. 27, 2023
(86) PCT No.: PCT/EP2023/070840

§ 371 Date: Feb. 12, 2025

(30) Foreign Application Priority Data
Aug. 18, 2022 (IT) 102022000017385

(51) Int. Cl. B65B 55/10; A61L 2/208; A61L 2/26; A61L 103/10; B65B 3/00; B65B 55/02

(52) U.S. Cl. CPC B65B 55/10; A61L 2/208; A61L 2/26; B65B 3/003; B65B 55/025; A61L 2103/10

(57) ABSTRACT

A feeding station for the decontamination of containers, which comprises a tunnel formed by a plurality of treatment chambers which are arranged side by side in succession along a container conveyance direction and in which a conveyor is provided, wherein each treatment chamber is subjected to an internal pressure higher than the atmospheric pressure by introducing air, gas or vapor and, at its entrance and its exit, is delimited by respective partitions which move between a passthrough position, in which the partitions leave open a respective passage opening of the container, and a position for maintaining the internal pressure, wherein the passage opening is reduced so as to allow a suction, through the residual opening, of said air, gas or vapor toward the outside.

The present invention relates to a feeding station for the decontamination of substantially identical containers to be fed in succession to a subsequent aseptic treatment station, for example a chamber for processing in a controlled atmosphere (also known as an “isolator”), or to an aseptic filling line (for example for pharmaceutical products), or to a station for the aseptic insertion of other vessels into the containers.

The containers that are particularly adapted to be used with the feeding station according to the invention are “nests” or “tubs”, i.e. trays or tubs that accommodate or are adapted to accommodate, preferably in an ordered and stabilized manner, smaller vessels such as vials, bottles, ampoules, cartridges, carpules®, test tubes and/or syringes, before and/or after filling. Other possible containers that are adapted to be used with the feeding station according to the invention are sterile bags.

Currently it is known to supply containers such as nests or tubs that are already decontaminated and isolated inside a sealed primary bag, in turn inserted into a sealed secondary bag. In order to introduce such containers into an aseptic treatment line or station, the operator manually removes the secondary bag and hangs the primary bag (which envelops the container) on a rack. A robot then cuts the primary bag and, while keeping the mouth still sealed using special jaws, brings it to the entrance of an aseptic isolator, into which the container is finally introduced, slipping it out from the primary bag.

A drawback of this conventional solution is the fact that it requires two bags to be used to isolate the container prior to its introduction into the aseptic treatment station. This, in addition to increasing costs and complicating the production of these two-bag packages, reduces the production speed of the aseptic treatment line or station into which the containers are introduced. Typically, with the technique described above, two containers per minute can be fed to the aseptic treatment station, notwithstanding the fact that the station can actually work at higher production speeds.

Furthermore, the above-mentioned “No-Touch” unbagging operation with robots is particularly complex, to the point that this has a major impact in terms of layout of the plant, considering that the dimensions of the plant that needs to be provided especially for unbagging the containers actually exceed the dimensions of the aseptic treatment station downstream, for example the station that carries out the filling.

Furthermore, in the conventional solution described above, there are still risks associated with the unbagging operation, such as contamination of the cleanrooms in which the process takes place, or of the actual container being conveyed.

The aim of the present invention is to provide a feeding station for feeding decontaminated containers to an aseptic treatment station, or line, that is capable of improving the known art in one or more of the above mentioned aspects.

Within this aim, an object of the invention is to make it possible to increase the production rate of the aseptic treatment station, or line.

Another object of the invention is to avoid the use at least of the outermost (secondary) bag to seal the containers before introducing them into the aseptic treatment station, or line.

Furthermore, the present invention sets out to overcome the drawbacks of the background art in a manner that is alternative to any existing solutions.

Another object of the invention is to provide a feeding station that is highly reliable, easy to implement and of low cost.

This aim and these and other objects which will become better apparent hereinafter are achieved by a feeding station according to claim 1, optionally provided with one or more of the characteristics of the dependent claims.

According to the invention, the feeding station for the decontamination of substantially identical containers while they are fed in succession to a subsequent station or line for aseptic treatment comprises a tunnel formed by a plurality of treatment chambers arranged side by side in succession along a container conveyance direction, conveyance means being provided in said treatment chambers and being adapted to convey the containers along said conveyance direction through said treatment chambers, characterized in that each treatment chamber is subjected to an internal pressure that is higher than the atmospheric pressure by introducing air, gas or vapor and, at its inlet and its outlet, said treatment chamber is delimited by respective partitions which can move between a crossing position, in which they leave open a respective passage opening of the container, and a position for maintaining said internal pressure, wherein said passage opening is reduced so as to allow a suction, through a residual opening, of said air, gas or vapor toward the outside of the treatment chamber in order to maintain said internal pressure.

The aims and objects of the invention are also achieved by an aseptic treatment line, comprising a loading line or entrance for loading containers, the feeding station arranged downstream of the loading line so as to receive said containers from the loading line or entrance, and an aseptic treatment station arranged downstream of the feeding station so as to receive the decontaminated containers, said aseptic treatment station comprising at least one assembly for filling the containers.

The aims and objects of the invention are also achieved by a method of decontamination of containers for pharmaceutical products while they are being conveyed through a feeding station toward an aseptic treatment station or line, said feeding station comprising a tunnel defined by a plurality of treatment chambers arranged in succession along a conveyance direction between an entrance and an exit, each treatment chamber being delimited by respective partitions respectively upstream and downstream with respect to said conveyance direction, said method comprising the steps of:
subjecting each treatment chamber to an internal pressure that is higher than the atmospheric pressure by introducing air, gas or vapor;
arranging the partitions in a passthrough position, so as to leave open a respective passage opening when the containers advance along the conveyance direction passing through said passage opening;
arranging the partitions in a position for maintaining said internal pressure, so as to reduce said passage opening to a residual opening when the containers are retained inside the respective chamber, said position for maintaining said internal pressure preventing the advancement or passage of the containers but allowing a suction of said air, gas or vapor from the respective treatment chamber to the outside, so as to maintain said internal pressure inside the respective chamber;
drawing by suction said air, gas or vapor from the respective treatment chamber to the outside so as to maintain said internal pressure inside the respective chamber.

Further characteristics and advantages of the invention will become better apparent from the detailed description that follows of a preferred, but not exclusive, embodiment of the feeding station according to the invention, which is illustrated for the purposes of non-limiting example in the accompanying drawings wherein:

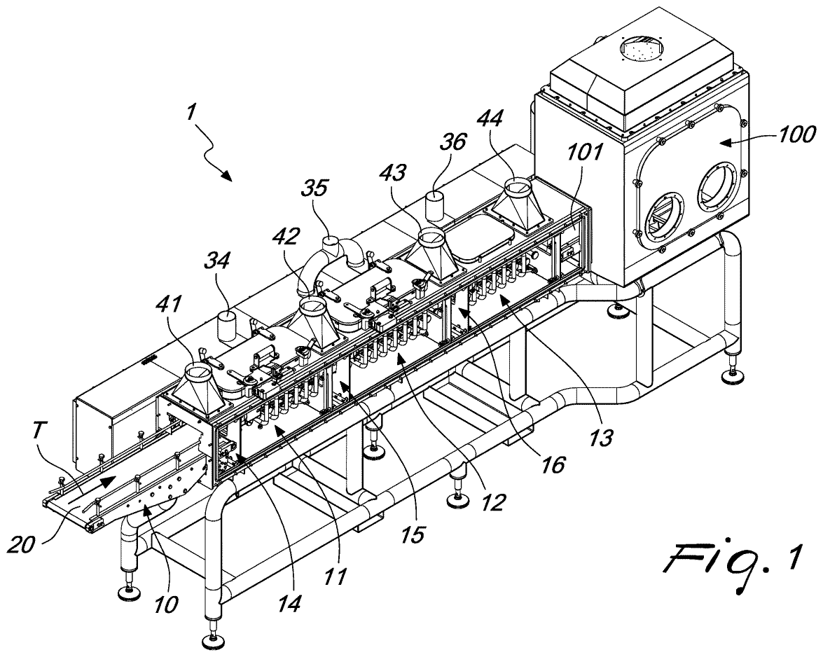

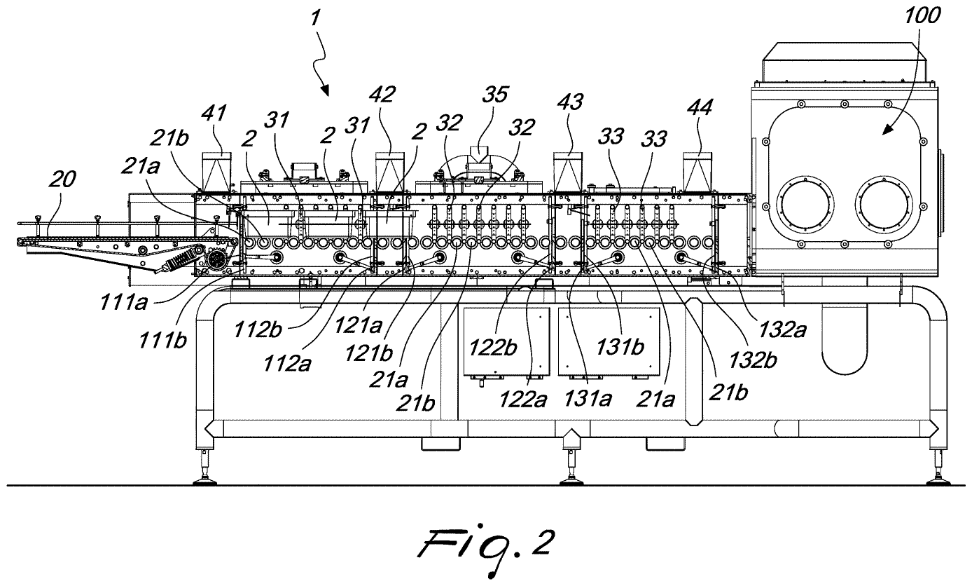

FIG. 1 is a perspective view of a feeding station according to the invention, in which the side panels have been removed to show the inside of the tunnel;

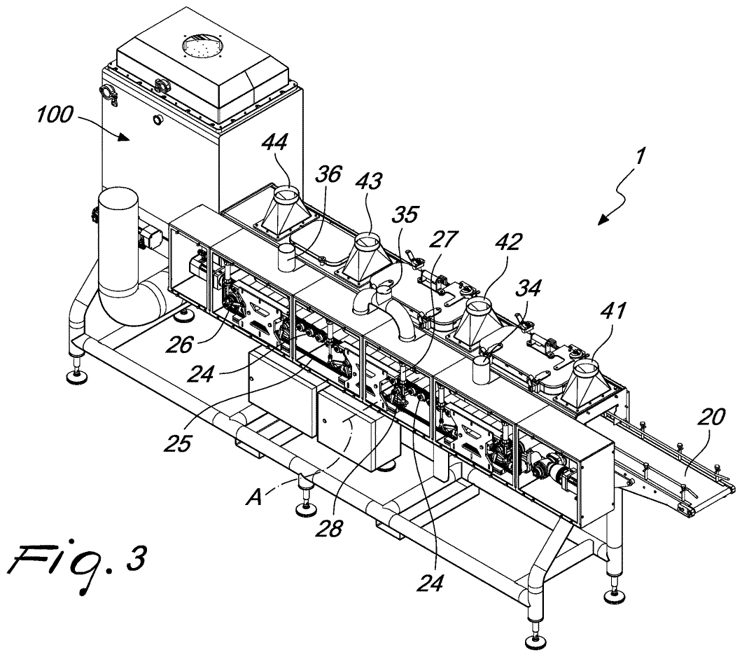

FIG. 2 is a side view of the feeding station of the previous figure, with some components removed for clarity;

FIG. 3 is another perspective view of the feeding station in the previous figures, seen from the side of the transmissions;

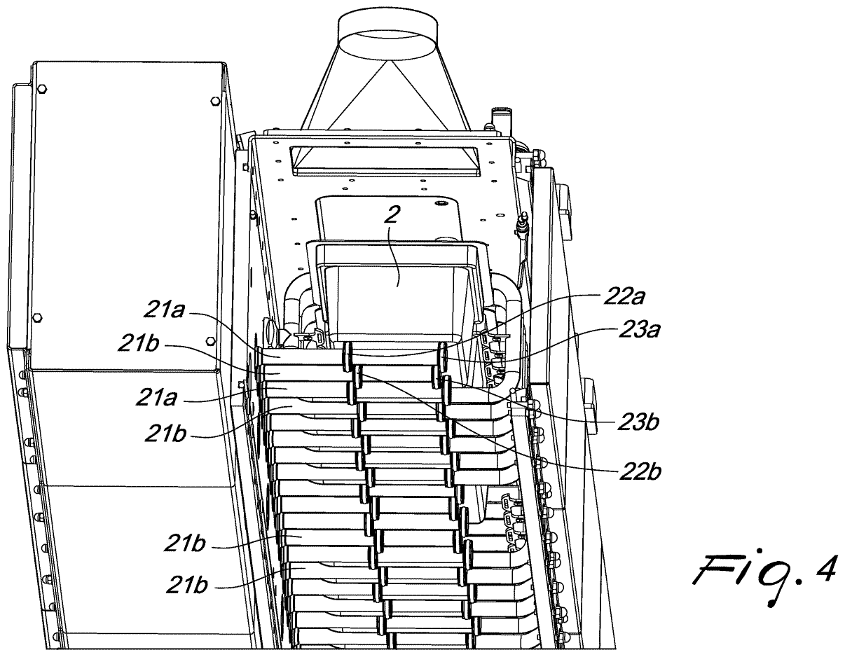

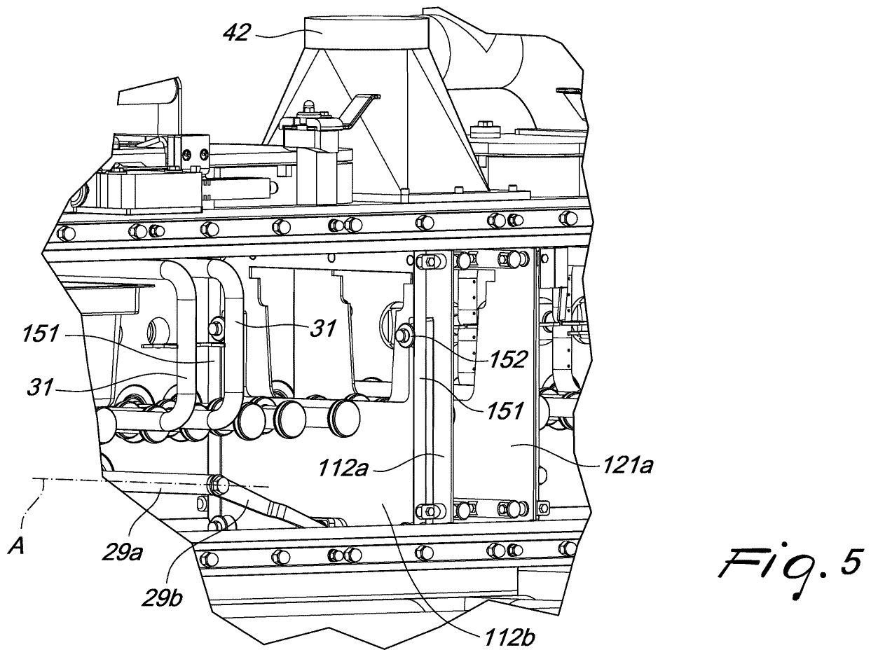

FIG. 4 is a detail seen from below of the conveyance means used in the feeding station in the previous figures;

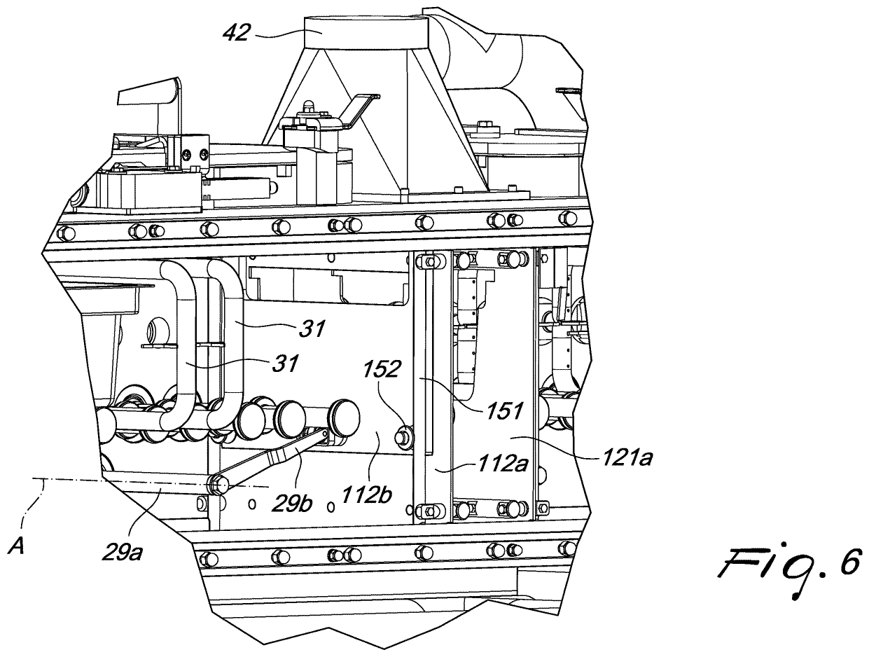

FIG. 5 shows an exit partition of one of the treatment chambers of the feeding station of FIG. 1, in the passthrough position;

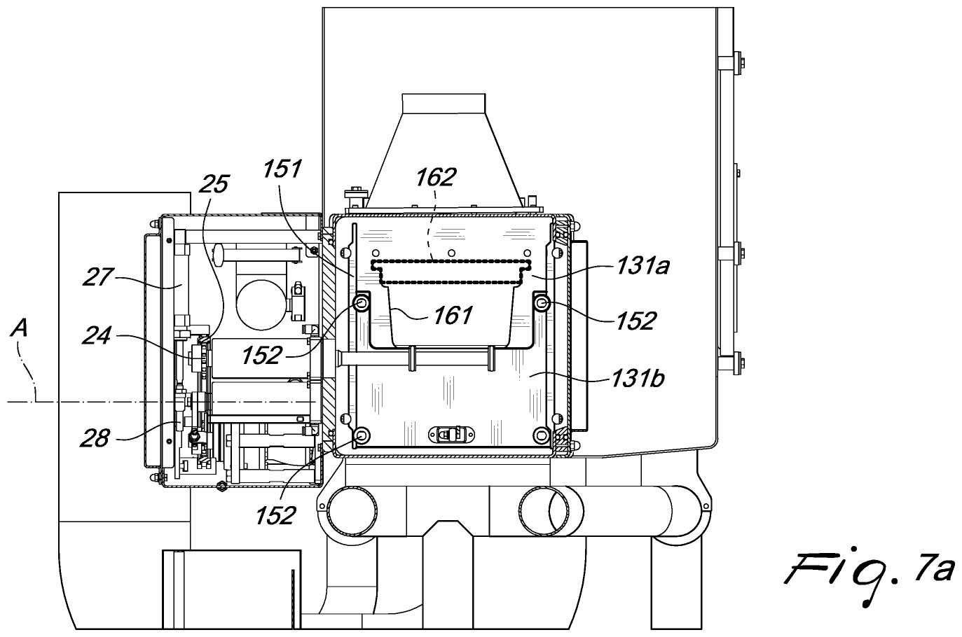

FIG. 6 shows the partition of the previous figure but in a position for maintaining the internal pressure;

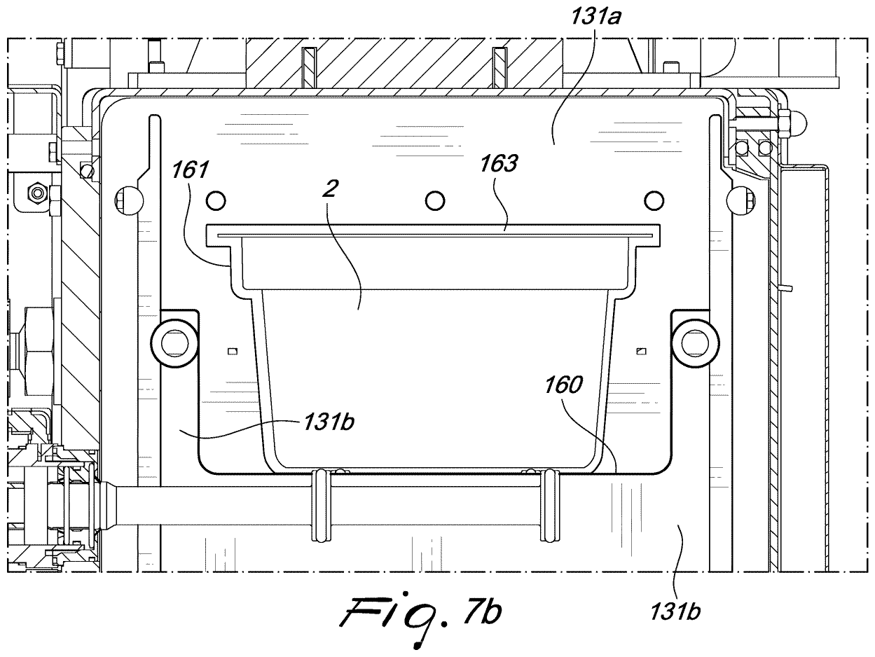

FIG. 7a is a front elevation view of a partition in the passthrough position;

FIG. 7b is a detail of the view of the previous figure, which also shows the bulk of the container on a plane at right angles to the conveyance direction;

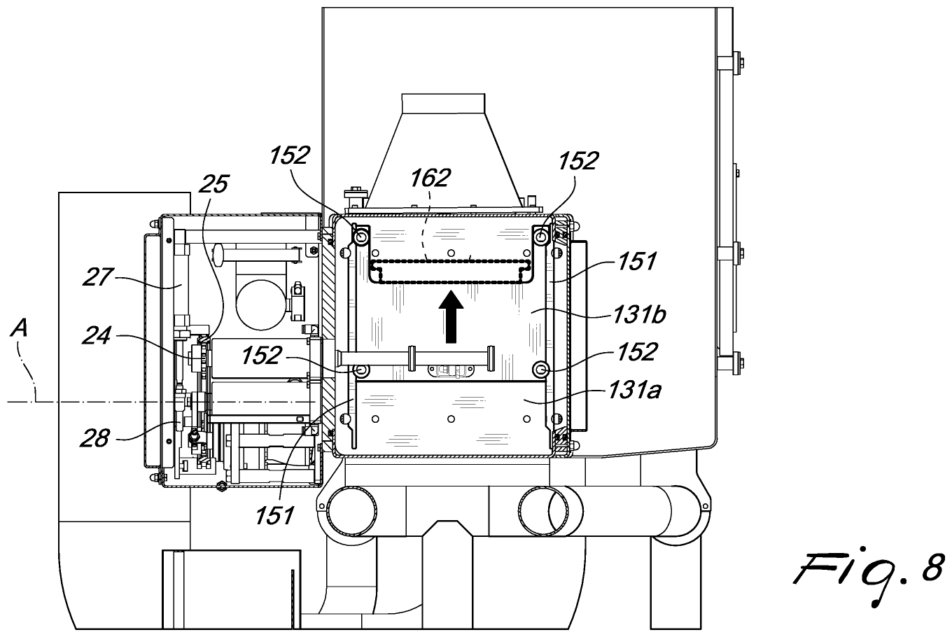

FIG. 8 is a front elevation view of the partition of FIG. 7a in the position for maintaining the pressure;

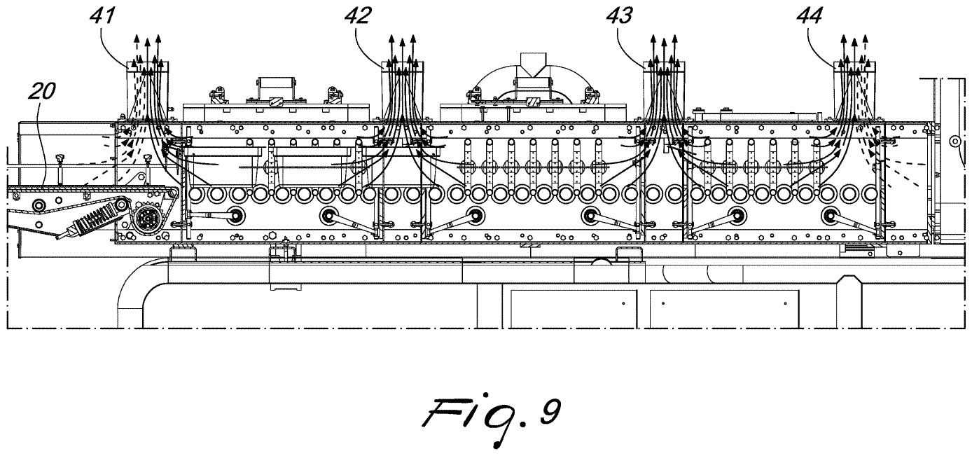

FIG. 9 shows the flows aspirated from the treatment chambers of the feeding station of FIG. 1;

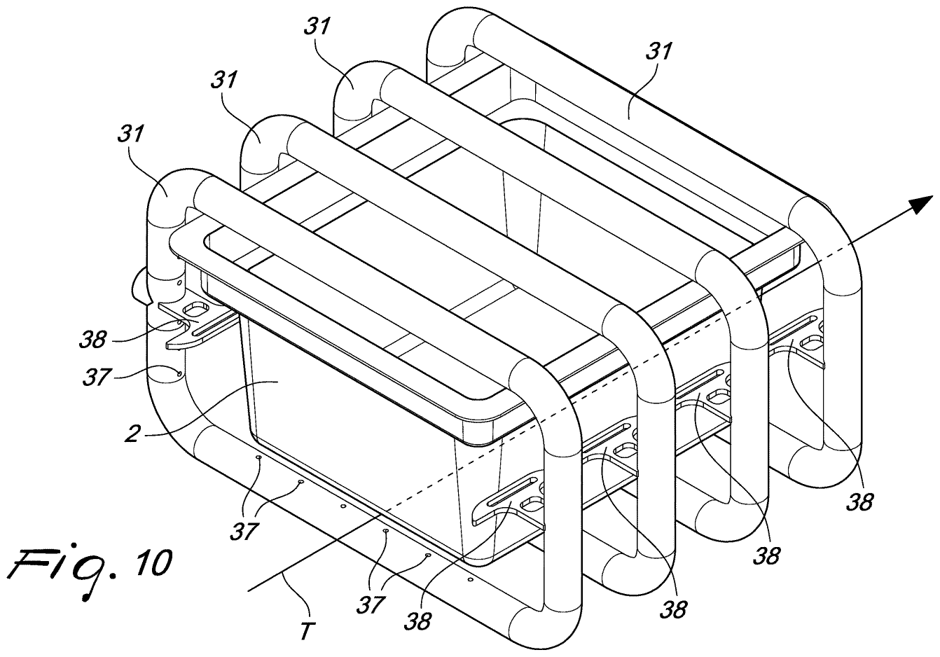

FIG. 10 is a detail view of a series of annular distribution units of one of the treatment chambers in the previous figures.

With reference to the figures, a feeding station according to the invention, generally designated by the reference numeral 1, is adapted for the decontamination of substantially mutually identical containers 2 (particularly for pharmaceutical products) which are fed or conveyed one after the other, in succession and with the same orientation, to a subsequent aseptic treatment station or line 100, for example a chamber for treatment in a controlled atmosphere (also known as an “isolator”). The aseptic treatment station 100 may comprise an assembly for filling the containers.

In the example shown, the containers 2 are tubs, i.e. the tubs specific to the pharmaceutical sector described above, and they are mutually identical in shape and dimensions. However, the feeding station 1 can be easily adapted to the decontamination of other containers, as long as they are substantially mutually identical and fed in a line with the same orientation, with respect to the conveyance direction T. The containers 2 can be individually supplied decontaminated and inside a single, respective sealed bag (to be opened before introducing the container into the feeding station 1), without the need for a secondary bag which contains the latter. The containers 2 can be made, for example, of polymeric material, of glass or of metal.

The feeding station 1 comprises a loading entrance or line 10, for example a conveyor belt 20 or a motorized roller conveyor, and a tunnel formed by a plurality of treatment chambers 11, 12, 13 which are arranged side by side in succession along a conveyance direction T of the containers 2 and which are closed gas-tight all around the conveyance direction T. Inside the treatment chambers 11-13 are conveyance means that are adapted to convey, with continuous motion, the containers 2 along the tunnel in the conveyance direction T and which are described below.

The treatment chambers 11, 12, 13 are kept at an internal pressure higher than the atmospheric pressure by introducing air, gas or vapor into each chamber.

Such internal pressure is preferably lower than the pressure inside the aseptic treatment station 100 downstream of the feeding station 1, in order to prevent any flows of contaminant agents toward the aseptic treatment station 100.

In particular, the overpressure applied in the treatment chambers 11, 12, 13 with respect to the atmospheric pressure can be less than 100 pascals, for example less than 25 pascals. For example, the pressure inside each one of the treatment chambers 11, 12, 13 is kept at a value comprised between 8 and 20 pascals of overpressure with respect to the atmospheric pressure, more preferably between 10 and 15 pascals above the atmospheric pressure. In the embodiment illustrated, the overpressure inside the treatment chambers 11 and 12 can be approximately 10 pascals, while the overpressure inside the treatment chamber 13 can be approximately 15 pascals and the overpressure inside the isolator 100 can be 25 pascals.

The treatment chambers consist preferably of a first, preparation chamber 11, a second, decontamination chamber 12, for example using vaporized hydrogen peroxide (VHP), and a third, rinsing chamber 13.

Air is introduced under pressure into the preparation chamber 11, such air being conveniently heated (and filtered) to increase the surface temperature of the containers 2 and so prevent the condensation of the decontaminant agent (for example VHP) in the subsequent decontamination chamber 12. The air can be introduced from a respective intake 34 connected to the discharge outlet of a respective pump (not shown) and can be distributed inside the treatment chamber 11 using a plurality of annular distribution units 31, which are connected to the entrance 34 and arranged side-by-side along the conveyance direction T so as to surround the conveyance means 21a-21b of the containers 2. Each distribution unit 31 has, all around the conveyance direction T i.e. all around the conveyance means, holes or nozzles 37 that are adapted to continuously spray the hot air in a direction substantially transverse to the conveyance direction T, so as to sweep over the containers 2 in transit.

Then, the decontaminant agent (gas or vapor) is introduced under pressure into the decontamination chamber 12 in order to decontaminate the surface of the containers 2 arriving from the preparation chamber 11. The decontaminant agent is preferably VHP (with concentrations comprised between 3000 and 20000 ppm) but, alternatively, decontaminant gases such as nitrogen dioxide or chlorine dioxide can be used.

In the chamber 12 too, there is a plurality of annular distribution units 32 which are connected to an intake 35 for the decontaminant gas or vapor and which are arranged side-by-side along the conveyance direction T so as to surround the conveyance direction T, i.e. the conveyance means 21a-21b for conveying the containers 2 within the chamber 12. Each distribution unit 32 has, all around the conveyance means 21a-21b, holes or nozzles 37 that are adapted to continuously spray the decontaminant gas or vapor in a direction substantially transverse to the conveyance direction T, so as to completely sweep over the surface of the containers 2 in transit.

Air under pressure, for example hot, is introduced into the rinsing chamber 13 to remove the residues of the decontaminant agent from the surface of the containers 2 arriving from the decontamination chamber 12. In the chamber 13 too, there is a plurality of annular distribution units 33 which are connected to an intake 36 for the air and which are arranged side-by-side along the conveyance direction T so as to surround the conveyance means 21a-21b for conveying the containers 2 within the chamber 13. Each distribution unit 33 has, all around the conveyance direction T, i.e. all around the conveyance means 21a-21b, holes or nozzles 37 that are adapted to continuously spray the air in a direction substantially transverse to the conveyance direction T, so as to completely sweep over the surface of the containers 2 in transit.

Upstream and downstream of each treatment chamber 11, 12, 13 there is a suction vent 41, 42, 43, 44 which is adapted to extract by suction, preferably continuously, the air or the vapor sprayed by the distribution units 31, 32, 33 and to thus keep the overpressure of each treatment chamber 11, 12, 13 substantially constant. For the rinsing chamber 13, the suction vent 44 can lead directly to the chamber 13, while the other vents 41-43 lead to a region of the tunnel that is outside the treatment chambers 11-12. This avoids the situation where the extracted flow could dilute the agents used inside one or more of the treatment chambers (for example VHP, as described below).

Between one treatment chamber 11, 12 and the next treatment chamber 12, 13, respectively, along the conveyance direction T, there is preferably a respective intermediate chamber 15, 16 which leads to a respective one of the vents 42 and 43 for the suction of the air or of the vapor from the treatment chamber upstream and/or downstream of the respective intermediate chamber 15, 16.

At the entrance and exit of each treatment chamber 11, 12, 13 along the conveyance direction T, there are respective partitions which can move, preferably vertically in a direction at right angles to the conveyance direction T of the containers 2. In particular, the preparation chamber 11 is delimited at the entrance and at the exit, respectively, by the movable partitions 111b and 112b. The decontamination 12 and rinsing 13 chambers are also delimited, respectively, by the movable partitions 121b-122b and 131b-132b.

The partitions 111b-112b, 121b-122b and 131b-132b can move between a passthrough position for containers, in which a respective passage opening 161 for the containers 2 is open, and a position for maintaining the internal pressure of the chamber (i.e. the overpressure generated in it), in which the above mentioned passage opening 161 is reduced and preferably is not entirely closed by at least one movable partition for each treatment chamber 11-13. In this manner, in the pressure-maintaining position of the movable partitions, a continuous suction is possible, through the residual opening 162, of the air or of the vapor from inside the respective treatment chamber 11, 12, 13 to outside the tunnel through the vents 41-43 which are outside of the treatment chambers, in order to keep the internal pressure of each treatment chamber substantially constant.

The movement of the partitions 111b-112b, 121b-122b and 131b-132b between the above-mentioned two positions is commanded through automatic detection of the transit of each container 2, for example obtained using a laser, and actuation means which are each associated with a respective one of the movable partitions.

The actuation means can be provided by way of an actuation cylinder 27, fixed in a box beside the tunnel, and a lever system for lifting/lowering a respective movable partition 111b-112b, 121b-122b, 131b-132b. A piston of the actuation cylinder 27 is hinged on a first lever 28, for example a rocker arm, which is pivoted on a rotation axis A. A second lever 29b, arranged inside the tunnel, is connected with one of its ends to the first lever 28 via a transmission shaft 29a which is coaxial to the rotation axis A, so that a rotation of the first lever 28 caused by the linear motion of the piston of the cylinder 27 is rigidly transmitted to the second lever 29b. A second end of the second lever 29b is connected to a respective movable partition 111b-112b, 121b-122b, 131b-132b so that a rotation of the second lever 29b is converted to a vertical translation of the movable partition between the passthrough position and the position for maintaining the internal pressure. For example, there can be a slot protruding from the movable partition and in which the second end of the lever 29b engages through a wheel.

According to a preferred aspect of the invention, the passage opening 161 which is, alternately, opened and partially obstructed by the respective movable partition 111b-112b, 121b-122b, 131b-132b can be defined by an opening which is provided in a respective fixed wall, 111a, 121a, 131a at the entrance and 112a, 122a, 132a at the exit, of each treatment chamber 11, 12, 13 and which can be passed through by each container 2 during the conveyance.

The opening on each one of the fixed walls 111a-112a, 121a-122a, 131a-132a has a shape that is substantially complementary to the transverse space occupation of the container 2 with respect to the conveyance direction T so that, while the container 2 is passing through the opening, the passage opening 161 is reduced by the transverse space occupation of the container 2 in transit.

Advantageously, the area of the planar interspace 163 between the edge of the passage opening 161 and the container 2 that is passing through the passage opening 161 is substantially equal to the area of the residual opening 162 of the passage opening 161 when the latter is reduced by the respective partition 111b-112b, 121b-122b, 131b-132b in the position for maintaining the internal pressure.

In this manner, at each entrance and exit of each treatment chamber 11-13, immediately before and immediately after the transit of each container 2, the movable partition is returned to the position for maintaining the pressure, while during the transit of the container through the passage opening 161, it is the container 2 itself that partially obstructs the passage opening 161 so as not to substantially depressurize the treatment chamber it enters or from which it exits. The container 2 that partially obstructs the passage opening 161 will leave the interspace 163 which still allows the suction of air or vapor through the vents 41-43 in order to maintain the internal pressure.

For this reason, the containers 2 are inserted at the entrance 20 of the tunnel in a row (i.e. one behind the other, along the conveyance direction T) and with a same, predefined orientation, so that the area of the above mentioned interspace 163 around the container is kept substantially constant for all the containers 2 in transit and can be determined so as to also define the area of the residual opening 162.

Each one of the movable partitions 111b-112b, 121b-122b, 131b-132b advantageously has a U-shaped recess and can move at right angles to the conveyance direction T so that the residual opening 162 of the passage opening is at least partially delimited by an edge 160 of the U-shaped recess. Since the movable partition (111b-112b, 121b-122b, 131b-132b) having such a U-shaped recess results to have a substantially “U” shape, the U-shaped recess will be also referred to as “U-shaped opening”. In particular, each movable partition 111b-112b, 121b-122b, 131b-132b can slide in a position parallel and adjacent to a respective one of the fixed walls 111a-112a, 121a-122a, 131a-132a so that the residual opening 162 of the passage opening 161 is partially delimited by the edge 160 of the U-shaped recess and partially by an edge of the opening of the fixed wall 111a-112a, 121a-122a, 131a-132a that defines the passage opening 161, as shown in FIG. 8.

To guide the vertical sliding of each movable partition, there can be vertical fixed rails 151 associated with the respective fixed wall 111a-112a, 121a-122a, 131a-132a, and guiding rollers 152 mounted on the movable partition 111b-112b, 121b-122b, 131b-132b and engaged on the vertical rails 151.

According to another advantageous aspect of the invention, the conveyance means for conveying the containers 2 through the tunnel comprise a roller conveyor 21a, 21b, in which the rollers are arranged in each treatment chamber 11-13 in alternation with the distribution units 31, 32, 33 along the conveyance direction T. Preferably, there is a respective annular distribution unit 31, 32, 33 between each pair of rollers.

The rollers 21a, 21b can be moved by means of a sprocket-and-chain system, in which a respective sprocket 24 keyed on one end of each roller 21a, 21b engages a transmission chain 25 which is common to all the sprockets 24 and is driven by a motor 26. The sprockets 24, the chain 25 and the motor 26 are mounted in the same box at the side of the tunnel in which the means for actuating the movable partitions and the connecting pipes between the distribution units 31-33 and the entrances 34-36 are mounted.

Each roller 21a, 21b comprises cylindrical enlargements 22a-23a, 22b-23b which are coaxial to the rollers and are adapted to engage by friction a bottom surface of each container 2 in order to make it advance along the conveyance direction T. For example, the cylindrical enlargements 22a-23a, 22b-23b can be surrounded by a respective O-ring or by another material adapted to cause a grip on the lower surface of the containers 2. In the embodiment illustrated, each roller comprises two cylindrical enlargements, mutually spaced apart along the axis of the roller.

Advantageously, the cylindrical enlargements 22a-23a, 22b-23b are mutually staggered along the conveyance direction T so that, during the conveyance, the same portion of the lower surface of the container 2 is not always engaged by the cylindrical enlargements. In this manner it is possible to effectively decontaminate the entire lower surface of the containers 2, with no parts being always shielded by the conveyance means.

In the embodiment illustrated, each roller 21a differs from the one directly upstream and downstream 21b in the mutual distance between the cylindrical enlargements 22a-23a, 22b-23b. Obviously other staggered arrangements of the cylindrical enlargements are possible, for example a same distance between the cylindrical enlargements of a same roller, but with a different axial position between one roller 21a and the immediately adjacent rollers 21b.

The operation of the feeding station according to the invention is evident from the foregoing description.

The containers 2 are placed continuously in a row one after the other on the loading entrance 20 by an operator or by a robot, after having been removed from the respective bag containing them. The orientation of the containers 2 on the entrance 20 is the same and is maintained along all of the tunnel of the feeding station 1. Optionally side guiding shoulders can be provided along the tunnel (for example plates 38 jutting inward on opposite sides of each one of the annular distribution units 31-33) to keep the containers 2 in the correct orientation, so that the (cross-sectional) outline of the containers is complementary to that of the passage openings 161 at the moment of their passing through.

In all the treatment chambers 11-13, an overpressure is maintained by way of the continuous introduction of air (into the chambers 11 and 13) and of decontaminant gas or vapor (into the chamber 12) and via the corresponding, continuous outflow through the vents 41-44. With the continuous rotation of the rollers 21a-21b, the containers 2 are made advance with continuous motion along all of the tunnel in the direction T.

Upon the arrival of each container 2 before the fixed wall 111a-121a-131a for entrance to each treatment chamber 11-13 and before the fixed wall 112a-122a-132a for exit from each treatment chamber 11-13, a (laser) sensor detects the passage of the container 2 and commands the movement of the corresponding movable partition 111b-121b-131b or 112b-122b-132b to the passthrough position. During the transit of the container 2, the contoured opening of the fixed wall 111a-121a-131a or 112a-122a-132a that is passed through, i.e. the corresponding passage opening 161, is substantially filled by the transverse space occupation of the container 2, leaving the interspace 163 and thus keeping the internal pressure of the corresponding treatment chamber, into/out of which the container 2 enters/exits, substantially constant with respect to the previous situation, i.e. the situation with the corresponding partition 111b-121b-131b or 112b-122b-132b in the position for maintaining the pressure.

As soon as the container 2 has passed through the opening of each fixed wall completely, the corresponding movable partition 111b-121b-131b or 112b-122b-132b returns to the position for maintaining the pressure, again by virtue of detection using a transit sensor (for example a laser sensor).

During the transit through each treatment chamber 11-13, the container 2 will undergo a surface heating (chamber 11), a decontamination (chamber 12) and a rinsing (chamber 13). The length of each treatment chamber and the speed of continuous conveyance are conveniently chosen on the basis of the time that is desired for the container to remain in the corresponding chamber to carry out the corresponding function.

Optionally, to remove plastic residues of the containers or other residues, all the chambers can be washed with a water jet or other washing agent at the end of the decontamination of a plurality of containers 2, for example by having, at the base of each chamber 11-15, washing nozzles connected to a source of water (or other washing liquid) under pressure, so as to spray it inside the respective chambers when the latter are inoperative or in overpressure.

In practice it has been found that the invention fully achieves the intended aim and objects. The feeding station according to the invention enables a continuous production at greater speeds (for example, 6 containers per minute), not least by virtue of the fact that the tunnel constitutes an open system, in which the treatment chambers are always in mutual communication, although with a variable passage opening.

The invention thus conceived is susceptible of numerous modifications and variations, all of which are within the scope of the appended claims. Moreover, all the details may be substituted by other, technically equivalent elements.

In practice the materials employed, provided they are compatible with the specific use, and the contingent dimensions and shapes, may be any according to requirements and to the state of the art.

The disclosures in Italian Patent Application No. 102022000017385 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1-15. (canceled)

16. A feeding station for the decontamination of containers for pharmaceutical products while the containers are being conveyed to an aseptic treatment station or line, said feeding station comprising a tunnel formed by a plurality of treatment chambers arranged in succession along a conveyance direction between an entrance and an exit; conveyance means adapted to convey the containers along said conveyance direction through said treatment chambers, in which each treatment chamber is configured to be subjected to an internal pressure higher than the atmospheric pressure by introducing air, gas or vapor, each treatment chamber being delimited by respective partitions respectively upstream and downstream with respect to the conveyance direction, wherein said partitions move between a pass-through position, in which the partitions leave open a respective passage opening in order to allow the container to pass through said passage opening, and a position for maintaining said internal pressure, in which said passage opening is reduced to a residual opening which is such as to prevent containers from passing through but to allow a suction of said air, gas or vapor from the respective treatment chamber to the outside, so as to maintain said internal pressure inside the respective chamber.

17. The feeding station according to claim 16, wherein said passage opening is provided on a respective fixed wall and has a shape that is substantially complementary to the transverse outline of the container with respect to the conveyance direction, so that the container almost completely occludes said passage opening while passing through it.

18. The feeding station according to claim 17, wherein, when said partition is in said pass-through position, between the passage opening and the container while passing through the passage opening, a space remains an area of which is substantially equal to an area of said residual opening.

19. The feeding station according to claim 16, wherein said movable partition has a U-shaped opening and moves at right angles to the conveyance direction so that said residual opening is at least partially delimited by an edge of said U-shaped opening.

20. The feeding station according to claim 19, wherein said partition slides in a position parallel and adjacent to a respective one of said fixed walls so that said residual opening is partially delimited by said edge of said U-shaped opening and partially by an edge of said passage opening on the respective fixed wall.

21. The feeding station according to claim 16, wherein between each treatment chamber and an adjacent treatment chamber along the conveyance direction there is an intermediate chamber which comprises an outlet for the suction of the air, gas or vapor from the treatment chamber upstream and/or downstream of the respective intermediate chamber with respect to the conveyance direction.

22. The feeding station according to claim 16, wherein each treatment chamber comprises a plurality of annular distribution units arranged side by side along the conveyance direction and adapted to completely surround said containers, each annular distribution unit being provided with holes or nozzles adapted to spray said air, gas or vapor against the containers.

23. The feeding station according to claim 22, wherein said conveyance means comprise a roller conveyor, having rollers comprising cylindrical enlargements which are coaxial to the rollers and are adapted to engage by friction a bottom surface of each container in order to make the container advance along the conveyance direction, said cylindrical enlargements being mutually offset on adjacent rollers along the conveyance direction.

24. The feeding station according to claim 23, wherein said annular distribution units are arranged in each treatment chamber in alternation with said rollers along the conveyance direction.

25. An aseptic treatment line, comprising a loading line or entrance for loading containers, a feeding station according to claim 16 arranged downstream of the loading line so as to receive said containers from the loading line or entrance, and an aseptic treatment station arranged downstream of the feeding station so as to receive decontaminated containers, said aseptic treatment station comprising at least one assembly for filling the containers.

26. A method of decontamination of containers for pharmaceutical products while the containers are being conveyed through a feeding station toward an aseptic treatment station or line, said feeding station comprising a tunnel defined by a plurality of treatment chambers arranged in succession along a conveyance direction between an entrance and an exit, each treatment chamber being delimited by respective partitions respectively upstream and downstream with respect to the conveyance direction, said method comprising the steps of:
subjecting each treatment chamber to an internal pressure that is higher than the atmospheric pressure by introducing air, gas or vapor;
arranging the partitions in a pass-through position, so as to leave open a respective passage opening when the containers advance along the conveyance direction passing through said passage opening;
arranging the partitions in a position for maintaining said internal pressure, so as to reduce said passage opening to a residual opening when the containers are retained inside the respective chamber, said position for maintaining said internal pressure preventing the advancement or passage of the containers but allowing a suction of said air, gas or vapor from the respective treatment chamber to the outside, so as to maintain said internal pressure inside the respective chamber;
drawing by suction said air, gas or vapor from the respective treatment chamber to the outside so as to maintain said internal pressure inside the respective chamber.

27. The method of decontamination according to claim 26, wherein said treatment chambers comprise, along the conveyance direction, a first preparation chamber into which hot air under pressure is introduced in order to heat said containers, a second decontamination chamber into which a decontaminating gas or vapor under pressure is introduced in order to decontaminate the surface of said containers, and a third rinsing chamber into which air under pressure is introduced in order to remove residues of decontaminant from the surface of the containers.

28. The method of decontamination according to claim 27, wherein, in said first preparation chamber and in said second decontamination chamber, an overpressure is maintained that is lower than the overpressure maintained in said third rinsing chamber.

29. The method of decontamination according to claim 27, wherein said decontaminating gas or vapor is vaporized hydrogen peroxide, or nitrogen dioxide or chlorine dioxide.

30. The method of decontamination according to claim 28, wherein said hydrogen peroxide has a concentration comprised between 3000 and 20000 ppm.
